(19) United States
(12) Patent Application Publication
(10) Pub. No.: US 2026/0000594 A1
(43) Pub. Date: Jan. 1, 2026

(54) COMPACT SHAMPOO COMPOSITION

(71) Applicant: THE PROCTER & GAMBLE COMPANY, Cincinnati, OH (US)

(72) Inventors: Eric Scott JOHNSON, Hamilton, OH (US); Kevin Patrick CHRISTMAS, Mason, OH (US)

(21) Appl. No.: 19/252,345

(22) Filed: Jun. 27, 2025

Related U.S. Application Data
(60) Provisional application No. 63/665,563, filed on Jun. 28, 2024.

(51) Int. Cl. A61K 8/44; A61K 8/04; A61Q 5/02

(52) U.S. Cl. CPC A61K 8/442; A61K 8/046; A61Q 5/02; A61K 2800/5428

(57) ABSTRACT

The present invention is directed to a compact, stable shampoo composition comprising: from about 18% to about 40%, by weight of the composition, of a surfactant system wherein the surfactant system comprises: i) from about 5% to about 40%, by weight of the composition, acyl glutamate; ii) from about 5% to about 40%, by weight of the composition, co-surfactant wherein the co-surfactant is selected from the group consisting of anionic surfactant, zwitterionic surfactant, and combinations thereof; wherein the shampoo composition is substantially free of sulfate-based surfactants; wherein the weight ratio of acyl glutamate to total surfactant is from about 0.30 to about 1; wherein the pH is from about 6 to about 8; wherein the composition has a viscosity of less than or equal to about 35 centipoise (cP).

### FIELD OF THE INVENTION

The present invention relates to shampoo compositions. More particularly a stable, compact shampoo composition that is substantially free of sulfate-based surfactants.

### BACKGROUND OF THE INVENTION

Some consumers desire a shampoo composition in a foam form. Given the low density of the foam, shampoo ingredients may be present at a higher concentration. For instance, compact shampoo compositions can contain a high concentration of surfactant, to deliver enough surfactant for proper cleaning and lather during use.

Shampoos also typically employ sulfate-based surfactant systems because of their effectiveness in generating high lather volume and good lather stability and cleaning. However, some consumers believe that sulfate-based surfactants, like sodium lauryl sulfate and sodium laureth sulfate, can be less gentle to the hair and skin, especially colored hair, as compared to compositions that are substantially free of sulfate-based surfactant systems.

Therefore, some consumers may prefer a shampoo composition that is substantially free of sulfate-based surfactant systems. However, it can be difficult to use these surfactants in traditional liquid shampoos because it is difficult to formulate a composition that has acceptable lather volume, cleansing, stability, and clarity. To improve lather volume and cleansing, shampoos containing sulfate-free surfactants typically have more surfactant than shampoos with sulfate-based surfactants, to achieve similar effectiveness.

The problems formulating with sulfate-free surfactants can be exacerbated when making a low viscosity compact formula. The high surfactant levels, especially high levels of acyl glutamate surfactants, can lead to product instability-For instance, before use, the sulfate-free surfactant can precipitate and/or form coacervates with the cationic polymers, which causes the shampoo to phase separate. This phase separation may be undesirable, particularly when low product viscosity is desired for dispensing the composition through a pump foamer.

Separate phase, if not stabilized, leads to non-uniform product usage or different performance throughout the life of the product. Sometimes it can lead to blocking of the orifice of a mechanical foam pump screen.

Therefore, there is a need for a phase stable, compact shampoo composition that is substantially free of sulfate-based surfactants.

### SUMMARY OF THE INVENTION

The present invention is directed to a compact, stable shampoo composition comprising: from about 18% to about 40%, by weight of the composition, of a surfactant system wherein the surfactant system comprises: i) from about 5% to about 40%, by weight of the composition, acyl glutamate; ii) from about 5% to about 40%, by weight of the composition, co-surfactant wherein the co-surfactant is selected from the group consisting of anionic surfactant, zwitterionic surfactant, and combinations thereof; wherein the shampoo composition is substantially free of sulfate-based surfactants; wherein the weight ratio of acyl glutamate to total surfactant is from about 0.30 to about 1; wherein the pH is from about 6 to about 8; wherein the composition has a viscosity of less than or equal to about 35 centipoise (cP).

### DETAILED DESCRIPTION OF THE INVENTION

While the specification concludes with claims particularly pointing out and distinctly claiming the invention, it is believed that the present disclosure will be better understood from the following description.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of.”

As used herein, the term “fluid” includes liquids, gels, emulsions, or suspensions.

As used herein, “mixtures” is meant to include a simple combination of materials and any compounds that may result from their combination.

As used herein, “molecular weight” or “M.Wt.” refers to the weight average molecular weight unless otherwise stated. Molecular weight is measured using industry standard method, gel permeation chromatography (“GPC”).

As used herein, “personal care composition” includes hair care products such as shampoos, conditioners, conditioning shampoos, hair colorants, as well as shower gels, liquid hand cleansers, facial cleansers, laundry detergent, dish detergent, and other surfactant-based liquid compositions.

As used herein, “substantially free” means less than 3%, less than 2%, less than 1%, less than 0.5%, less than 0.25%, less than 0.1%, less than 0.05%, less than 0.01%, less than 0.001%, and/or free of. As used herein, “free of” means 0%.

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Where amount ranges are given, these are to be understood as being the total amount of said ingredient in the composition, or where more than one species fall within the scope of the ingredient definition, the total amount of all ingredients fitting that definition, in the composition. For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the shampoo composition.

### Shampoo Composition

As will be described herein, stable, compact shampoo compositions are disclosed that are substantially free of sulfate-based surfactants. The compact shampoo can have a low liquid phase viscosity so it can be delivered through mechanical pump foamer.

It can be difficult to make shampoo compositions that are substantially free of sulfate-based surfactants because it can be difficult to formulate a composition that has acceptable lather volume, cleansing, stability, and clarity. These problems can be exacerbated when making a compact formula, which has higher surfactant levels. One significant problem is formulating a shampoo composition that is phase stable. It was surprisingly found that the weight ratio of glutamate to total surfactant is important for phase stability.

The shampoo compositions may have a viscosity of less than or equal to about 35 centipoise (cP), may have a viscosity of less than about 35 centipose (cP), may have a viscosity of less than about 25 centipoise (cP), as determined by the Cone/Plate Viscosity Measurement or Cup and Bob Viscosity Measurement, described hereafter. The shampoo composition may have a viscosity of from 1 to 35 centipoise (cP), from about 5 to about 30 centipoise (cP), from about 5 to about 25 centipoise (cP) and from about 10 (centipoise) cP to about 20 centipoise (cP), as determined by the Cone/Plate Viscosity Measurement or Cup and Bob Viscosity Measurement, described hereafter.

The shampoo compositions can be phase stable and may be substantially free of a thickener. Non-limiting examples of thickeners can include acrylate polymers and co-polymer such as Carbopol® SF-1, xanthan gum, and combinations thereof.

The shampoo compositions can have a pH from about-from about 5 to about 8, from about from about 5 to about 7, from about 6.5 to about 7.5, from about 6.2 to about 7.2, and from about 6 to about 7.

### Surfactant

The shampoo compositions described herein can include one or more surfactants in the surfactant system and the one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a shampoo composition can include anionic moieties to allow for the formation of a coacervate with a cationic polymer. The surfactant can be selected from anionic surfactants, amphoteric surfactants, zwitterionic surfactants, non-ionic surfactants, and combinations thereof.

Shampoo compositions typically employ sulfate-based surfactant systems (such as, but not limited to, sodium lauryl sulfate) because of their effectiveness in lather production, stability, clarity and cleansing. The foamed shampoo compositions described herein can be substantially free of sulfate-based surfactants.

The concentration of the surfactant in the composition should be sufficient to provide the desired cleaning and lather performance. The shampoo composition can comprise a total surfactant level of from about 10% to about 50%, by weight, from about 15% to about 48%, by weight, from about 18% to about 45%, by weight, from about 20% to about 40%, from about 18% to about 40%, by weight, from about 20% to about 35%, from about 20% to about 30%, and/or from about 21% to about 26%.

Suitable surfactants that are substantially free of sulfates can include isethionates, sulfonates, sulfosuccinates, sulfoacetates, acyl glucosides, acyl glycinates, acyl sarcosinare, acyl glutamates, acyl alaninates, glucamide, glucose carboxylates, amphoacetates, taurates, other acyl aminoacids, betaines, sultaines, and/or phosphate esters. Suitable surfactants that are substantially free of sulfates can contain carboxylic acids.

The foamed shampoo composition can contain an anionic surfactant. The composition can comprise a total anionic surfactant level from about 5% to about 40%, by weight, from about 8% to about 35%, by weight, from about 10% to about 35%, by weight, from about 10% to about 30%, by weight, from about 10% to about 25%, by weight, and/or from about 15% to about 22%, by weight. The composition can comprise a total anionic surfactant level from about 15% to about 30%, from about 15% to about 25%, and/or from about 18% to about 22%.

The surfactant system can contain from about 10% to about 100% anionic surfactant, by weight, from about 20% to about 90% anionic surfactant, by weight of the surfactant system, and/or from about 30% to about 90%, and/or from about 40% to about 88%, and/or from about 50% to about 85%, and/or from about 60% to about 85%. The surfactant system can contain from about 50% to about 100% anionic surfactant, by weight, from about 60% to about 100% anionic surfactant, by weight of the surfactant system, and/or from about 66% to about 100%, and/or from about 70% to about 98%, and/or from about 75% to about 97%, and/or from about 80% to about 92%.

The composition can contain an anionic surfactant and the anionic surfactant can be a glutamate, for instance an acyl glutamate. The composition can comprise an acyl glutamate level from about 2% to about 22%, by weight, from about 3% to about 19%, by weight, 4% to about 17%, by weight, and/or from about 5% to about 16%, by weight. The composition can contain less than 20%, less than 15%, by weight, acyl glutamate, less than 13%, less than 12%, less than 10%, less than 8%, less than 6%. The composition can contain more acyl glutamate than any other surfactant.

The ratio of acyl glutamate to total surfactant can be less than 1.0. less than 0.90, less than 0.80, less than 0.70, less than 0.60, less than 0.50, less than 0.4, less than or equal to 0.35. The ratio of acyl glutamate to total surfactant can be from about 0.05 to about 1, from about 0.1 to about 1, from about 0.15 to about 1, from about 0.20 to about 1, from about 0.25 to about 1, from about 0.30 to about 1, from about 0.3 to about 0.7.

Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The composition can contain one or more co-surfactants selected from the group consisting of an anionic co-surfactant, zwitterionic surfactant, non-ionic surfactant, and combinations thereof. The composition can contain a primary surfactant that can be an acyl glutamate and an anionic co-surfactant.

The anionic co-surfactant can be selected from the group consisting of isethionates, sarcosinates, sulfosuccinates, sulfonates, sulfoacetates, acyl glycinates, acyl alaninates, glucose carboxylates, alkyl ether carboxylates, alkyl amphoacetates, acyl taurates, and mixture thereof. The anionic co-surfactant level can be from about 0% to about 30%, by weight, from about 2% to about 25%, by weight, from about 5% to about 20%, by weight, and/or from about 7% to about 15%, by weight. The anionic co-surfactant level from about 0% to about 20%, by weight, from about 3% to about 18%, by weight, from about 5% to about 15%, by weight, and/or from about 7% to about 12%, by weight. The composition can comprise an anionic co-surfactant level from about 5% to about 15%, by weight.

Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinate, diamyl ester of sodium sulfosuccinic acid, dihexyl ester of sodium sulfosuccinic acid, dioctyl esters of sodium sulfosuccinic acid, and combinations thereof.

Non-limiting examples of sulfonates can include alpha olefin sulfonates, linear alkylbenzene sulfonates, sodium laurylglucosides hydroxypropylsulfonate and combination thereof.

Non-limiting examples of sulfoacetates can include sodium lauryl sulfoacetate, ammonium lauryl sulfoacetate and combination thereof.

Non-limiting example of acyl glycinates can include sodium cocoyl glycinate, sodium lauroyl glycinate and combination thereof.

Non-limiting example of acyl alaninates can include sodium cocoyl alaninate, sodium lauroyl alaninate, sodium N-dodecanoyl-1-alaninate and combination thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of alkylamphoacetates can include sodium cocoyl amphoacetate, sodium lauroyl amphoacetate and combination thereof.

Non-limiting example of acyl taurates can include sodium methyl cocoyl taurate, sodium methyl lauroyl taurate, sodium methyl oleoyl taurate and combination thereof.

The surfactant system may further comprise one or more zwitterionic surfactants and the zwitterionic surfactant can be a co-surfactant selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

Examples of betaine zwitterionic surfactants can include coco dimethyl carboxymethyl betaine, cocoamidopropyl betaine (CAPB), cocobetaine, lauryl amidopropyl betaine (LAPB), oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

The zwitterionic surfactant can comprise cocamidopropyl betaine (CAPB), lauramidopropyl betaine (LAPB), and combinations thereof.

The foamed shampoo composition can comprise a zwitterionic surfactant level from about 0% to about 15%, by weight, from about 1% to about 12%, by weight, from about 2% to about 10%, by weight, and/or from about 5% to about 7.5%, by weight. The surfactant system can contain from about 0% to about 40% zwitterionic surfactant, by weight of the surfactant system, and/or from about 0% to about 35%, and/or from about 5% to about 20%.

The surfactant system can have a weight ratio of anionic surfactant to zwitterionic surfactant from about 1:1 to about 15:1, from about 3:2 to about 11:1, from about 4:1 to about 11:1, from about 1:1 to about 3:1.

The surfactant system can have a weight ratio of anionic surfactant to zwitterionic surfactant greater than 1:1, greater than 3:2, greater than 3:1, and/or greater than 5:1.

The surfactant system may further comprise one or more non-ionic surfactants and the non-ionic surfactant can be selected from the group consisting alkyl polyglucoside, alkyl glycoside, acyl glucamide and mixture thereof. Non-limiting examples of alkyl glucosides can include decyl glucoside, cocoyl glucoside, lauroyl glucoside and combination thereof. The non-ionic surfactants may be in the range of from about 3% to about 10%, from about 4% to about 8%, and from about 5% to about 7%.

Non-limiting examples of acyl glucamide can include lauroyl/myristoyl methyl glucamide, capryloyl/caproyl methyl glucamide, lauroyl/myristoyl methyl glucamide, cocoyl methyl glucamide and combinations thereof.

### Liquid Carrier

Inclusion of an appropriate quantity of a liquid carrier can facilitate the formation of a shampoo composition having an appropriate liquid viscosity and rheology. A shampoo composition can include, by weight of the composition, about 50% to about 95%, of a liquid carrier, about 60% to about 85%, about 65% to about 80%, about 68% to about 78%, and/or about 70% to about 77%.

A liquid carrier can be water, or can be a miscible mixture of water and organic solvent. A liquid carrier can be water with minimal or no significant concentrations of organic solvent, except as otherwise incidentally incorporated into the composition as minor ingredients of other essential or optional components. Suitable organic solvents can include water solutions of lower alkyl alcohols and polyhydric alcohols. Useful lower alkyl alcohols include monohydric alcohols having 1 to 6 carbons, such as ethanol and isopropanol. Exemplary polyhydric alcohols include propylene glycol, dipropylene glycol, hexylene glycol, glycerin, and propane diol.

### Cationic Polymer

A shampoo composition can include a cationic polymer to allow formation of a coacervate, particularly formation of a coacervate during use. As can be appreciated, the cationic charge of a cationic polymer can interact with an anionic charge of a surfactant to form the coacervate. Suitable cationic polymers can include: a cationic guar polymer, a cationic non-guar galactomannan polymer, a cationic starch polymer, a cationic copolymer of acrylamide monomers and cationic monomers, a synthetic, non-crosslinked, cationic polymer, which may or may not form lyotropic liquid crystals upon combination with the detersive surfactant, and a cationic cellulose polymer.

A cationic polymer can be included by weight of the shampoo composition at about 0.01% to about 2%, about 0.05% to about 1%, about 0.1% to about 0.8%, and/or from about 0.1% to about 0.5%. Cationic polymers can have cationic charge densities of about 0.9 meq/g or more, about 1.2 meq/g or more, and about 1.5 meq/g or more. However, cationic charge density can also be about 7 meq/g or less and/or about 5 meq/g or less. The charge densities can be measured at the pH of intended use of the shampoo composition. (e.g., at about pH 3 to about pH 9; or about pH 4 to about pH 8). The average molecular weight of cationic polymers can generally be between about 1,000 and 2 million, between about 5,000 and about 1 million, and between about 10,000 and about 0.5 million. Low molecular weight cationic polymers can be preferred. Low molecular weight cationic polymers can have greater translucency in the liquid carrier of a shampoo composition. Suitable cationic polymers can include Polyquaternium-6 with a charge density of about 6.2 meq/g and a M.Wt. of about 223,000 g/mole available from Clariant, Polyquaternium-76 with a charge density of about 1.6 meq/g and a M.Wt. of about 1.1 million g/mole.

### Cationic Guar Polymer

The cationic polymer can be a cationic guar polymer, which is a cationically substituted galactomannan (guar) gum derivative. Suitable guar gums for guar gum derivatives can be obtained as a naturally occurring material from the seeds of the guar plant. As can be appreciated, the guar molecule is a straight chain mannan which is branched at regular intervals with single membered galactose units on alternative mannose units. The mannose units are linked to each other by means of b(1-4) glycosidic linkages. The galactose branching arises by way of an a(1-6) linkage. Cationic derivatives of the guar gums can be obtained through reactions between the hydroxyl groups of the polygalactomannan and reactive quaternary ammonium compounds. The degree of substitution of the cationic groups onto the guar structure can be sufficient to provide the requisite cationic charge density described above.

A cationic guar polymer can have a weight average molecular weight (“M.Wt.”) of less than about 1 million g/mol, and can have a charge density from about 0.05 meq/g to about 2.5 meq/g. Cationic guar suitable can have a weight average molecular weight (“M.Wt.”) of less than about 0.5 million g/mol.

A shampoo composition can include from about 0.01% to less than about 0.7%, by weight of the shampoo composition of a cationic guar polymer, from about 0.05% to about 0.6%, from about 0.1% to about 0.50%, by weight, and/or from about 0.1% to about 0.4%, by weight.

The cationic guar polymer can be formed from quaternary ammonium compounds which conform to general Formula II:

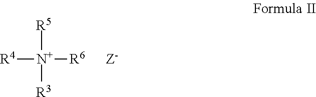

wherein where R³, R⁴ and R⁵ are methyl or ethyl groups; and R⁶ is either an epoxyalkyl group of the general Formula III:

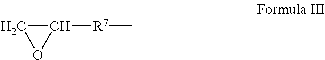

or R⁶ is a halohydrin group of the general Formula IV:

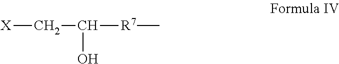

wherein R⁷ is a C₁ to C₃ alkylene; X is chlorine or bromine, and Z is an anion such as Cl—, Br—, I— or HSO₄—.

A cationic guar polymer can conform to the general formula V:

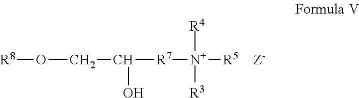

wherein R⁸ is guar gum; and wherein R⁴, R⁵, R⁶ and R⁷ are as defined above; and wherein Z is a halogen. A cationic guar polymer can conform to Formula VI:

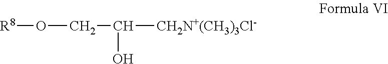

wherein R⁸ is guar gum.

Suitable cationic guar polymers can also include cationic guar gum derivatives, such as guar hydroxypropyltrimonium chloride. Suitable examples of guar hydroxypropyltrimonium chlorides can include the Jaguar® series commercially available from Solvay S. A., Hi-Care™ Series from Rhodia, and N-Hance™ and AquaCat™ from Ashland Inc. Jaguar® Optima has a charge density of about 1.25 meg/g and a M. Wt. of about 500,000 g/moles. Jaguar® C-500 has a charge density of 0.8 meq/g and a M.Wt. of 500,000 g/mole; Hi-Care™ 1000 has a charge density of about 0.7 meq/g and a M.Wt. of about 600,000 g/mole; N-Hance™ 3269, N-Hance™ 3270 and N-Hance 3271 have a charge density of about 0.7 meq/g and a M.Wt. of about 425,000 g/mole; AquaCat™ PF618 and AquaCat™ CG518 have a charge density of about 0.9 meq/g and a M.Wt. of about 50,000 g/mole. N-Hance™ BF-13 and N-Hance™ BF-17 are borate (boron) free guar polymers. N-Hance™ BF-13 has a charge density of about 1.1 meq/g and M.W.t of about 800,000 and N-Hance™ BF-17 has a charge density of about 1.7 meq/g and M.W.t of about 800,000.

### Cationic Non-Guar Galactomannan Polymer

A cationic polymer can be a galactomannan polymer derivative. A suitable galactomannan polymer can have a mannose to galactose ratio of greater than 2:1 on a monomer to monomer basis and can be a cationic galactomannan polymer derivative or an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers can be present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and can be affected by climate. Non-Guar Galactomannan polymer derivatives can have a ratio of mannose to galactose of greater than 2:1 on a monomer to monomer basis. Suitable ratios of mannose to galactose can also be greater than 3:1 or greater than 4:1. Analysis of mannose to galactose ratios is well known in the art and is typically based on the measurement of the galactose content.

The gum for use in preparing the non-guar galactomannan polymer derivatives can be obtained from naturally occurring materials such as seeds or beans from plants. Examples of various non-guar galactomannan polymers include Tara gum (3 parts mannose/1 part galactose), Locust bean or Carob (4 parts mannose/1 part galactose), and Cassia gum (5 parts mannose/1 part galactose).

A non-guar galactomannan polymer derivative can have a M. Wt. from about 1,000 g/mol to about 10,000,000 g/mol, and a M.Wt. from about 5,000 g/mol to about 3,000,000 g/mol.

The shampoo compositions described herein can include galactomannan polymer derivatives which have a cationic charge density from about 0.5 meq/g to about 7 meq/g. The galactomannan polymer derivatives can have a cationic charge density from about 1 meq/g to about 5 meq/g. The degree of substitution of the cationic groups onto the galactomannan structure can be sufficient to provide the requisite cationic charge density. A non-limiting example of non-guar galactomannan cationic polymer can be cassia cassia hydroxypropyltrimonium chloride known as ClearHance™ C available from Ashland.

A galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general Formulas II to VI, as defined above.

Cationic non-guar galactomannan polymer derivatives formed from the reagents described above can be represented by the general Formula VII:

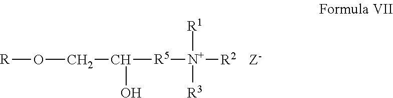

wherein R is the gum. The cationic galactomannan derivative can be a gum hydroxypropyltrimethylammonium chloride, which can be more specifically represented by the general Formula VIII:

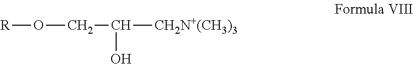

The galactomannan polymer derivative can be an amphoteric galactomannan polymer derivative having a net positive charge, obtained when the cationic galactomannan polymer derivative further comprises an anionic group.

A cationic non-guar galactomannan can have a ratio of mannose to galactose which is greater than about 4:1, a M.Wt. of about 100,000 g/mol to about 500,000 g/mol, a M.Wt. of about 50,000 g/mol to about 400,000 g/mol, and a cationic charge density from about 1 meq/g to about 5 meq/g, and from about 2 meq/g to about 4 meq/g.

Shampoo compositions can include at least about 0.05% of a galactomannan polymer derivative by weight of the composition. The shampoo compositions can include from about 0.05% to about 2%, by weight of the composition, of a galactomannan polymer derivative.

### Cationic Copolymer of an Acrylamide Monomer and a Cationic Monomer

A shampoo composition can include a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

Examples of suitable cationic polymers can include:
(i) an acrylamide monomer of the following Formula IX:

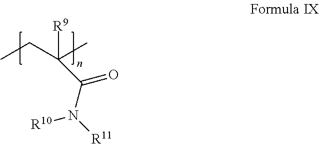

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula X:

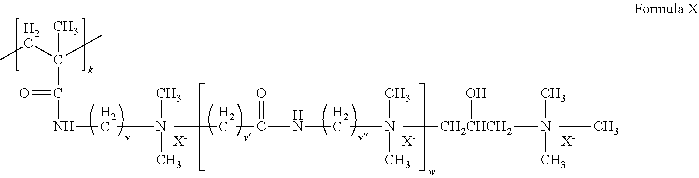

where k=1, each of v, v′, and v″ is independently an integer of from 1 to 6, w is zero or an integer of from 1 to 10, and X⁻ is an anion.

A cationic monomer can conform to Formula X where k=1, v=3 and w=0, z=1 and X⁻ is Cl⁻ to form the following structure (Formula XI):

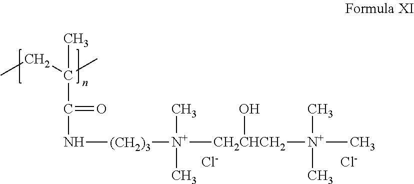

As can be appreciated, the above structure can be referred to as diquat.

A cationic monomer can conform to Formula X wherein v and v″ are each 3, v′=1, w=1, y=1 and X⁻ is Cl⁻, to form the following structure of Formula XII:

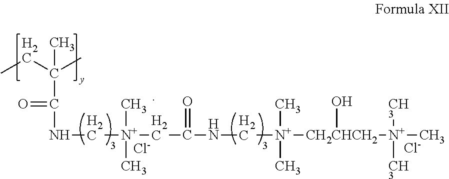

The structure of Formula XII can be referred to as triquat.

The acrylamide monomer can be either acrylamide or methacrylamide.

The cationic copolymer can be AM:TRIQUAT which is a copolymer of acrylamide and 1,3-Propanediaminium, N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT can have a charge density of 1.6 meq/g and a M.Wt. of 1.1 million g/mol.

The cationic copolymer can include an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can include a cationic monomer selected from the group consisting of: trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters can be cationized esters of the (meth)acrylic acid containing a quaternized N atom. Cationized esters of the (meth)acrylic acid containing a quaternized N atom are quaternized dialkylaminoalkyl (meth)acrylates with C₁ to C₃ in the alkyl and alkylene groups. The cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom can be dimethylaminoethyl acrylate, which is quaternized with an alkyl halide, or with methyl chloride or benzyl chloride or dimethyl sulfate (ADAME-Quat). The cationic monomer when based on (meth)acrylamides are quaternized dialkylaminoalkyl (meth)acrylamides with C₁ to C₃ in the alkyl and alkylene groups, or dimethylaminopropylacrylamide, which is quaternized with an alkyl halide, or methyl chloride or benzyl chloride or dimethyl sulfate.

The cationic monomer based on a (meth)acrylamide can be a quaternized dialkylaminoalkyl (meth)acrylamide with C₁ to C₃ in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate.

The cationic monomer can be a hydrolysis-stable cationic monomer. Hydrolysis-stable cationic monomers can be, in addition to a dialkylaminoalkyl (meth)acrylamide, any monomer that can be regarded as stable to the OECD hydrolysis test. The cationic monomer can be hydrolysis-stable and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with chloride methyl (ADAME-Q) and 3-dimethylammoniumpropyl (meth)acrylamide quaternized with methyl chloride (DIMAPA-Q). The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, from about 1.1 meq/g to about 2.3 meq/g, from about 1.2 meq/g to about 2.2 meq/g, from about 1.2 meq/g to about 2.1 meq/g, from about 1.3 meq/g to about 2.0 meq/g, and from about 1.3 meq/g to about 1.9 meq/g.

The cationic copolymer can have a M.Wt. from about 100 thousand g/mol to about 2 million g/mol, from about 300 thousand g/mol to about 1.8 million g/mol, from about 500 thousand g/mol to about 1.6 million g/mol, from about 700 thousand g/mol to about 1.4 million g/mol, and from about 900 thousand g/mol to about 1.2 million g/mol.

The cationic copolymer can be a trimethylammoniopropylmethacrylamide chloride-N-Acrylamide copolymer, which is also known as AM:MAPTAC. AM:MAPTAC can have a charge density of about 1.3 meq/g and a M.Wt. of about 1.1 million g/mol. The cationic copolymer is AM:ATPAC. AM:ATPAC can have a charge density of about 1.8 meq/g and a M.Wt. of about 1.1 million g/mol.

### Synthetic Polymers

A cationic polymer can be a synthetic polymer that is formed from:
i) one or more cationic monomer units, and optionally
ii) one or more monomer units bearing a negative charge, and/or
iii) a nonionic monomer,
   wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers which have the structure of Formula XIII:

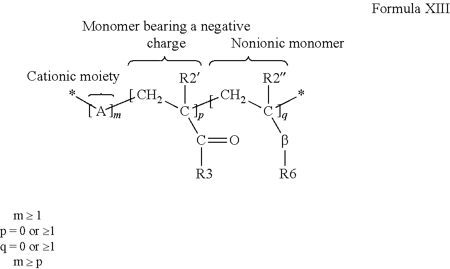

where A, may be one or more of the following cationic moieties:

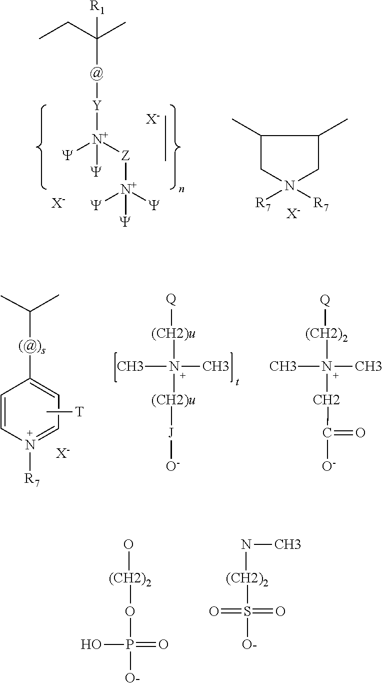

where @=amido, alkylamido, ester, ether, alkyl or alkylaryl;
where Y=C1-C22 alkyl, alkoxy, alkylidene, alkyl or aryloxy;
where y=C1-C22 alkyl, alkyloxy, alkyl aryl or alkyl arylox;
where Z=C1-C22 alkyl, alkyloxy, aryl or aryloxy;
where R1=H, C1-C4 linear or branched alkyl;
where s=0 or 1, n=0 or ³ 1;
where T and R7=C1-C22 alkyl; and
where X−=halogen, hydroxide, alkoxide, sulfate or alkylsulfate.

Where the monomer bearing a negative charge is defined by R2′=H, C₁-C₄ linear or branched alkyl and R³ is:
where D=O, N, or S;
where Q=NH₂ or O;
where u=1-6;
where t=0-1; and
where J=oxygenated functional group containing the following elements P, S, C.

Where the nonionic monomer is defined by R2″=H, C₁-C₄ linear or branched alkyl, R6=linear or branched alkyl, alkyl aryl, aryl oxy, alkyloxy, alkylaryl oxy and b is defined as

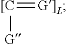

and
where G′ and G″ are, independently of one another, O, S or N—H and L=0 or 1.

Suitable monomers can include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of suitable cationic monomers can include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers can include quaternary monomers of formula —NR₃⁺, wherein each R can be identical or different, and can be a hydrogen atom, an alkyl group comprising 1 to 10 carbon atoms, or a benzyl group, optionally carrying a hydroxyl group, and including an anion (counter-ion). Examples of suitable anions include halides such as chlorides, bromides, phosphates, citrates, formates, and acetates.

Suitable cationic monomers can also include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride. Additional suitable cationic monomers can include trimethyl ammonium propyl (meth)acrylamido chloride.

Examples of monomers bearing a negative charge include alpha ethylenically unsaturated monomers including a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge can include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers can include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers can also include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers can be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non-limiting examples of suitable counterions can include halides (e.g., chlorine, fluorine, bromine, iodine).

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer can return the hair to a generally virgin-like, healthier state. Lyotropic liquid crystals are formed by combining the synthetic cationic polymers described herein with the aforementioned anionic detersive surfactant component of the shampoo composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in PCT Patent App. No. WO 94/06403 which is incorporated by reference. The synthetic polymers described herein can be formulated in a stable shampoo composition that provides improved conditioning performance, with respect to damaged hair.

Cationic synthetic polymers that can form lyotropic liquid crystals have a cationic charge density of from about 2 meq/gm to about 7 meq/gm, and/or from about 3 meq/gm to about 7 meq/gm, and/or from about 4 meq/gm to about 7 meq/gm. The cationic charge density is about 6.2 meq/gm. The polymers also have a M. Wt. of from about 1,000 to about 5,000,000, and/or from about 10,000 to about 2,000,000, and/or from about 100,000 to about 2,000,000.

Cationic synthetic polymers that provide enhanced conditioning and deposition of benefit agents but do not necessarily form lytropic liquid crystals can have a cationic charge density of from about 0.7 meq/gm to about 7 meq/gm, and/or from about 0.8 meq/gm to about 5 meq/gm, and/or from about 1.0 meq/gm to about 3 meq/gm. The polymers also have a M.Wt. of from about 1,000 g/mol to about 5,000,000 g/mol, from about 10,000 g/mol to about 2,000,000 g/mol, and from about 100,000 g/mol to about 2,000,000 g/mol.

### Cationic Cellulose Polymer

Suitable cationic polymers can be cellulose polymers. Suitable cellulose polymers can include salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose can include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose can include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

Techniques for analysis of formation of complex coacervates are known in the art. For example, microscopic analyses of the compositions, at any chosen stage of dilution, can be utilized to identify whether a coacervate phase has formed. Such coacervate phase can be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated by reference.

### Optional Ingredients

As can be appreciated, shampoo compositions described herein can include a variety of optional components to tailor the properties and characteristics of the composition. As can be appreciated, suitable optional components are well known and can generally include any components which are physically and chemically compatible with the essential components of the shampoo compositions described herein. Optional components should not otherwise unduly impair product stability, aesthetics, or performance. Individual concentrations of optional components can generally range from about 0.001% to about 10%, by weight of a shampoo composition. Optional components can be further limited to components which will not impair the clarity of a translucent shampoo composition.

Optional components may include, but are not limited to, conditioning agents (including hydrocarbon oils, fatty esters, silicones), cationic polymers, anti-dandruff actives, chelating agents, and natural oils such as sun flower oil or castor oil. Additional suitable optional ingredients include but are not limited to perfumes, perfume microcapsules, colorants, particles, anti-microbials, foam boosters, anti-static agents, propellants, self-foaming agents, pH adjusting agents and buffers, preservatives, pearlescent agents, solvents, diluents, anti-oxidants, vitamins and combinations thereof.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

### Silicone Conditioning Agent

A shampoo composition can include a silicone conditioning agent. Suitable silicone conditioning agents can include volatile silicone, non-volatile silicone, or combinations thereof. A silicone conditioning agent, the agent can be included from about 0.01% to about 10% active silicone material, by weight of the composition, from about 0.1% to about 8%, from about 0.1% to about 5%, and/or from about 0.2% to about 3%. Examples of suitable silicone conditioning agents, and optional suspending agents for the silicone, are described in U.S. Reissue Pat. No. 34,584, U.S. Pat. Nos. 5,104,646, and 5,106,609, each of which is incorporated by reference herein. Suitable silicone conditioning agents can have a viscosity, as measured at 25° C., from about 20 centistokes (“csk”) to about 2,000,000 csk, from about 1,000 csk to about 1,800,000 csk, from about 50,000 csk to about 1,500,000 csk, and from about 100,000 csk to about 1,500,000 csk.

The dispersed silicone conditioning agent particles can have a volume average particle diameter ranging from about 0.01 micrometer to about 50 micrometer. For small particle application to hair, the volume average particle diameters can range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), which is incorporated herein by reference.

Silicone emulsions suitable for the shampoo compositions described herein can include emulsions of insoluble polysiloxanes prepared in accordance with the descriptions provided in U.S. Pat. No. 4,476,282 and U.S. Patent Application Publication No. 2007/0276087 each of which is incorporated herein by reference. Suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm. The silicone emulsion can further include an additional emulsifier together with the anionic surfactant.

Other classes of silicones suitable for the shampoo compositions described herein can include i) silicone fluids, including silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly crosslinked polymeric siloxane systems; vi) high refractive index silicones, having refractive index of at least 1.46, and vii) mixtures thereof.

The silicone conditioning agent can be a silicone emulsion having particles size less than about 10 microns, less than 1 microns and less than 0.1 microns.

### Organic Conditioning Materials

The conditioning agent of the shampoo compositions described herein can also include at least one organic conditioning material such as oil or wax, either alone or in combination with other conditioning agents, such as the silicones described above. The organic material can be non-polymeric, oligomeric or polymeric. The organic material can be in the form of an oil or wax and can be added in the personal care formulation neat or in a pre-emulsified form. Suitable examples of organic conditioning materials can include: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Scalp Care Active

Scalp care actives suitable for use in shampoo compositions of the present invention may include hydroxy pyridones, such as piroctone olamine (commercially available as Octopirox®), ciclopirox, rilopirox, and MEA-Hydroxyoctyloxypyridinone; azoles, such as climbazole, ketoconazole, itraconazole, econazole, and elubiol; kerolytic agents, such as salicylic acid and other hydroxy acids. A scalp care active may include strobilurins such as azoxystrobin and metal chelators such as 1,10-phenanthroline; polyvalent metal salts of pyrithione, non-limiting examples include zinc pyrithione (ZPT) and copper pyrithione; sulfur, and selenium sulfide.

The scalp care active may be present in an amount from about 0.01% to 10%, from about 0.1% to about 9%, from about 0.25% to 8%, and from about 0.5% to 6%. The scalp care active can be surfactant soluble and thus surfactant soluble scalp care active.

### Chelating Agents

The shampoo composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. Nos. 5,747,440. 5,284,972 and 5,747,440 are each incorporated by reference herein. Suitable chelants can further include histidine.

Levels of an EDDS chelant or histidine chelant in the shampoo compositions can be low. For example, an EDDS chelant or histidine chelant can be included at about 0.01%, by weight. Above about 10% by weight, formulation and/or human safety concerns can arise. The level of an EDDS chelant or histidine chelant can be at least about 0.05%, by weight, at least about 0.1%, by weight, at least about 0.25%, by weight, at least about 0.5%, by weight, at least about 1%, by weight, or at least about 2%, by weight, by weight of the shampoo composition.

### Product Form

The shampoo compositions may be presented in typical hair care formulations. They may be in the form of solutions, dispersion, emulsions, foams, and other delivery mechanisms. The composition can be a low viscosity or viscous liquid that can be applied to wet hair, then massaged into the hair, and then rinsed out.

The shampoo composition in the form of a foam can have a density of from about 0.02 g/cm³ to about 0.3 g/cm³. from about 0.02 g/cm³ to about 0.2 g/cm³, from about 0.025 g/cm³ to about 0.18 g/cm³, from about 0.05 g/cm³ to about 0.18 g/cm³, from about 0.1 g/cm³ to about 0.18 g/cm³. The density can be measured Foam Density & Foam Volume Method, described hereafter.

### Foam Dispenser

The shampoo composition may be stored and dispensed from a mechanical foam dispenser. Non-limiting examples of suitable pump dispensers include those described in WO 2004/078903, WO 2004/078901, and WO 2005/078063 and may be supplied by Albea (60 Electric Ave., Thomaston, CT 06787 USA) or Rieke Packaging Systems (500 West Seventh St., Auburn, Indiana 46706).

The shampoo composition may be stored and dispensed from a squeeze foam dispenser. An example of squeeze foamer is EZ′R available from Albéa.

The shampoo composition and/or the dispenser may be free or substantially free of a propellant, for instance aerosol propellants.

### Cone/Plate Viscosity Measurement

The viscosities of the examples are measured by a Cone/Plate Controlled Stress Brookfield Rheometer RSX, by Brookfield Engineering Laboratories, Stoughton, MA. The cone used (Spindle C75-1) has a diameter of 75 mm and lo angle. The liquid viscosity is determined using a steady state flow experiment at constant shear rate of 2 s-1 and at temperature of 26.7° C. The sample size is 2.5 ml and the total measurement reading time is 3 minutes.

### Cup and Bob Viscosity Measurement

The viscosities of the examples are measured by a TA Instruments Discovery HR-3 Rheometer, by TA Instruments, New Castle, DE. The method employes a Peltier concentric cylinder system, using a DIN rotor geometry (radius of 14 mm and height of 42 mm) and standard cup (rad=15 mm) The liquid viscosity is determined using a flow sweep experiment at shear rate range of 0.1-500 s-1, 10 points/decade, 1% Tolerance, and at temperature of 25° C. The sample size is 25 ml.

### Foam Density & Foam Volume

Foam density is measured by placing a 60 ml graduated cylinder onto a mass balance, tarring the mass of the beaker and then dispensing product from the container into the 60 ml graduated cylinder until the volume of the foam is above the rim of the vessel. The foam is made level with the top of the cylinder by scraping a spatula across it within 10 seconds of dispensing the foam above the rim of the vessel. The resulting mass of the 60 ml of foam is then divided by the volume (60) to determine the foam density in units of g/ml.

Foam volume may be measured by placing a weigh boat onto a mass balance, tarring the mass of the weigh boat and then dispensing the desired amount of product from the container. The grams of foam dispensed may be determined and then divided by the density of foam as determined from the Foam Density methodology to reach a volume of foam in ml or cm³.

### EXAMPLES

The following are non-limiting examples of the shampoo composition described herein. The examples were prepared by conventional techniques and included adding the ingredients one by one and mixing until homogeneous or dissolved and adding heat as necessary to dissolve particular ingredients. It will be appreciated that other modifications of the present invention within the skill of those in the shampoo formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

TABLE 1
Comparative Example and Shampoo Composition Examples 1-4

| | Comparative | Examples | | | |
|---|---|---|---|---|---|
| | Example | 1 | 2 | 3 | 4 |
| Disodium Laureth | 9.35 | — | — | — | — |
| Sulfosuccinate¹ | | | | | |
| Lauramidopropyl Betaine² | 7.78 | 7.70 | 7.70 | 7.70 | 7.43 |
| Disodium Cocoyl Glutamate³ | 6.32 | 11.75 | 8.36 | 15.76 | 8.07 |
| Sodium C10-15 Pareth | — | 4.01 | — | — | — |
| Sulfate⁴ | | | | | |
| Sodium Cocoyl Isethionate⁵ | — | — | — | — | — |
| Sodium Lauroyl Sarcosinate⁶ | — | — | — | — | 7.19 |
| Decyl Glucoside⁷ | — | — | 7.44 | — | — |
| Sodium Lauryl Sulfate⁸ | — | — | — | — | — |
| Linoleamidopropyl PG- | — | — | — | — | — |
| Dimonium Chloride | | | | | |
| Phosphate⁹ | | | | | |
| Stearamidopropyl | — | — | — | — | — |
| Dimethylamine¹⁰ | | | | | |
| Propylene Glycol¹¹ | — | — | — | — | 3.86 |
| Dipropylene Glycol¹² | — | — | — | — | 1.14 |
| Sodium Xylene Sulfonate¹³ | — | — | — | — | 0.93 |
| Glycerin¹⁴ | 2.84 | 2.84 | 2.84 | 2.84 | 2.75 |
| Polyquat-76¹⁵ | 0.20 | 0.20 | 0.20 | 0.20 | 0.19 |
| Sodium Benzoate¹⁶ | 0.25 | 0.25 | 0.25 | 0.25 | 0.24 |
| Sodium Salicylate¹⁷ | - | 0.25 | 0.25 | 0.25 | 0.24 |
| DiSodium EDTA¹⁸ | 0.14 | 0.15 | 0.14 | 0.14 | 0.14 |
| Methylchloroisothiazolinone/ | 0.0602 | — | — | — | — |
| Methylisothiazolinone¹⁹ | | | | | |
| Piroctone Olamine²⁰ | 0.80 | 0.81 | 0.80 | 0.81 | 0.78 |
| Fragrance | 1.70 | 1.69 | 1.70 | 1.72 | 1.65 |
| Citric Acid²¹ | 0.50 | 1.60 | 0.76 | 2.26 | 1.72 |
| Water | QS | QS | QS | QS | QS |
| pH | 6.41 | 5.79 | 6.39 | 5.63 | 5.5 |
| Viscosity (cP) | 13* | 130* | 27* | 67* | 74* |
| Pumpable? | Yes | Yes | Yes | Yes | Yes, but |
| | | | | | foam is |
| | | | | | watery |
| Pump Force (Avg. Peak Load | 43.889 | 56.245 | 48.955 | 42.469 | 60.635 |
| (N)) | | | | | |
| Pump Output (Avg. | 1.252 | 1.258 | 1.188 | 1.175 | 1.184 |
| Dose/Pump (g)) | | | | | |
| Foam Density (g/ml) | 0.1175 | 0.1276 | 0.1158 | 0.1824 | 0.2428 |
| Appearance | Slight haze, | Pale | Pale | hazy, | Transparent, |
| | translucent, | yellow | yellow | pale | light |
| | pale yellow | and | and | yellow, | yellow |
| | | transparent | transparent | with | |
| | | | | some | |
| | | | | small | |
| | | | | white | |
| | | | | particles | |

TABLE 2
Examples 5-9 of Shampoo Composition Examples

| | Examples | | | | |
|---|---|---|---|---|---|
| | 5 | 6 | 7 | 8 | 9 |
| Disodium Laureth | — | — | — | — | — |
| Sulfosuccinate¹ | | | | | |
| Lauramidopropyl Betaine² | 7.79 | 7.70 | 7.71 | 7.70 | 7.71 |
| Disodium Cocoyl Glutamate³ | 8.37 | 8.37 | 8.36 | 8.38 | 8.36 |
| Sodium C10-15 Pareth | — | — | — | — | — |
| Sulfate⁴ | | | | | |
| Sodium Cocoyl Isethionate⁵ | — | 7.44 | — | — | — |
| Sodium Lauroyl Sarcosinate⁶ | — | — | 7.45 | — | — |
| Decyl Glucoside⁷ | — | — | — | — | — |
| Sodium Lauryl Sulfate⁸ | 7.45 | — | — | 7.45 | — |
| Linoleamidopropyl PG- | — | — | — | — | 7.44 |
| Dimonium Chloride | | | | | |
| Phosphate⁹ | | | | | |
| Stearamidopropyl | — | — | — | — | — |
| Dimethylamine¹⁰ | | | | | |
| Propylene Glycol¹¹ | — | — | — | — | — |
| Dipropylene Glycol¹² | 6.00 | — | — | — | — |
| Sodium Xylene Sulfonate¹³ | 1.00 | — | — | — | — |
| Glycerin¹⁴ | 2.85 | 2.85 | 2.85 | 2.85 | 2.85 |
| Polyquat-76¹⁵ | 0.20 | 0.20 | 0.20 | 0.20 | 0.20 |
| Sodium Benzoate¹⁶ | 0.25 | 0.25 | 0.25 | 0.25 | 0.24 |
| Sodium Salicylate¹⁷ | 0.25 | 0.25 | 0.25 | 0.25 | 0.24 |
| DiSodium EDTA¹⁸ | 0.14 | 0.14 | 0.15 | 0.14 | 0.15 |
| Methylchloroisothiazolinone/ | — | — | — | — | — |
| Methylisothiazolinone¹⁹ | | | | | |
| Piroctone Olamine²⁰ | 0.81 | 0.80 | 0.81 | 0.80 | 0.80 |
| Fragrance | 1.69 | 1.72 | 1.70 | 1.68 | 1.71 |
| Citric Acid²¹ | 1.17 | 1.30 | 1.67 | 1.08 | 1.47 |
| Water | QS | QS | QS | QS | QS |
| pH | 5.82 | 5.62 | 5.58 | 6.04 | 5.59 |
| Viscosity (cP) | 72* | NA; | 663\hat{ } | 5030\hat{ } | 914\hat{ } |
| | | phase | | | |
| | | split | | | |
| Pumpable? | Yes, but | No | No | No | No |
| | foam is | | | | |
| | watery | | | | |
| Pump Force (Avg. Peak Load | 66.127 | | | | |
| (N)) | | | | | |
| Pump Output (Avg. | 1.170 | | | | |
| Dose/Pump (g)) | | | | | |
| Foam Density (g/ml) | 0.1946 | | | | |
| Appearance | Light | Thick, | Pale | Transparent, | Opaque |
| | yellow | phase | yellow, | slight | |
| | and | split, gel | transparent | haze | |
| | transparent | like | | | |
| | upon | material | | | |
| | equilibration | on the | | | |
| | | bottom. | | | |

TABLE 3
Examples 10-13 of Shampoo Composition Examples

| | Examples | | | |
|---|---|---|---|---|
| | 10 | 11 | 12 | 13 |
| Disodium Laureth | — | — | — | — |
| Sulfosuccinate¹ | | | | |
| Lauramidopropyl Betaine² | 7.71 | 7.70 | 7.70 | 7.70 |
| Disodium Cocoyl Glutamate³ | 12.31 | 8.36 | 8.36 | 8.36 |
| Sodium C10-15 Pareth | — | — | — | — |
| Sulfate⁴ | | | | |
| Sodium Cocoyl Isethionate⁵ | — | — | — | — |
| Sodium Lauroyl Sarcosinate⁶ | — | — | 7.45 | — |
| Decyl Glucoside⁷ | — | — | — | 7.44 |
| Sodium Lauryl Sulfate⁸ | — | — | — | — |
| Linoleamidopropyl PG- | — | 7.44 | — | — |
| Dimonium Chloride | | | | |
| Phosphate⁹ | | | | |
| Stearamidopropyl | 3.51 | — | — | — |
| Dimethylamine¹⁰ | | | | |
| Propylene Glycol¹¹ | — | 5.32 | 4.00 | — |
| Dipropylene Glycol¹² | — | — | — | 5.00 |
| Sodium Xylene Sulfonate¹³ | — | — | — | — |
| Glycerin¹⁴ | 2.85 | 2.84 | 2.85 | 2.84 |
| Polyquat-76¹⁵ | 0.20 | 0.20 | 0.20 | 0.20 |
| Sodium Benzoate¹⁶ | 0.25 | 0.24 | 0.24 | 0.25 |
| Sodium Salicylate¹⁷ | 0.25 | 0.24 | 0.25 | 0.25 |
| DiSodium EDTA¹⁸ | 0.14 | 0.14 | 0.14 | 0.14 |
| Methylchloroisothiazolinone/ | — | — | — | — |
| Methylisothiazolinone¹⁹ | | | | |
| Piroctone Olamine²⁰ | 0.80 | 0.80 | 0.80 | 0.80 |
| Fragrance | 1.69 | 1.70 | 1.71 | 1.70 |
| Citric Acid²¹ | 2.37 | 1.60 | 1.78 | 0.76 |
| Water | QS | QS | QS | QS |
| pH | 6.68 | 5.51 | 5.50 | 6.49 |
| Viscosity (cP) | 2737\hat{ } | 216* | 273* | 20\hat{ }\hat{ } |
| Pumpable? | No | No | No | Yes |
| Pump Force (Avg. Peak Load | | | | |
| (N)) | | | | |
| Pump Output (Avg. | | | | |
| Dose/Pump (g)) | | | | |
| Foam Density (g/ml) | | | | |
| Appearance | Opaque | Opaque | Transparent | Transparent |

TABLE 4
Examples 14-17 of Shampoo Composition Examples

| | Examples | | | |
|---|---|---|---|---|
| | 14 | 15 | 16 | 17 |
| Disodium Laureth Sulfosuccinate¹ | — | — | — | — |
| Lauramidopropyl Betaine² | 7.70 | 7.70 | 7.70 | 7.70 |
| Disodium Cocoyl Glutamate³ | 8.36 | 11.50 | 8.36 | 8.36 |
| Sodium C10-15 Pareth Sulfate⁴ | — | — | — | — |
| Sodium Cocoyl Isethionate⁵ | — | — | — | — |
| Sodium Lauroyl Sarcosinate⁶ | — | — | — | — |
| Decyl Glucoside⁷ | 7.44 | 4.30 | 7.44 | 7.44 |
| Sodium Lauryl Sulfate⁸ | — | — | — | — |
| Linoleamidopropyl PG-Dimonium | — | — | — | — |
| Chloride Phosphate⁹ | | | | |
| Stearamidopropyl | — | — | — | — |
| Dimethylamine¹⁰ | | | | |
| Propylene Glycol¹¹ | 5.00 | 5.00 | — | — |
| Dipropylene Glycol¹² | — | — | — | — |
| Sodium Xylene Sulfonate¹³ | — | — | — | — |
| Glycerin¹⁴ | 2.84 | 2.84 | 2.84 | 2.84 |
| Polyquat-76¹⁵ | 0.20 | 0.20 | 0.20 | 0.20 |
| Sodium Benzoate¹⁶ | 0.25 | 0.25 | 0.25 | 0.25 |
| Sodium Salicylate¹⁷ | 0.25 | 0.25 | 0.25 | 0.25 |
| DiSodium EDTA¹⁸ | 0.14 | 0.14 | 0.14 | 0.14 |
| Methylchloroisothiazolinone/ | — | — | — | — |
| Methylisothiazolinone¹⁹ | | | | |
| Piroctone Olamine²⁰ | 0.80 | 0.80 | 0.80 | 0.80 |
| Fragrance | 1.70 | 1.70 | 1.30 | 1.70 |
| Menthol²³ | — | — | 0.40 | — |
| Citric Acid²¹ | 0.76 | 0.76 | 0.76 | 0.76 |
| Salicylic Acid²² | — | — | — | 1.80 |
| Water | QS | QS | QS | QS |
| pH | 6.56 | 6.70 | 6.5 | 6.5-7.0 |
| Viscosity (cP) | 22\hat{ }\hat{ } | 13\hat{ }\hat{ } | | |
| Pumpable? | Yes | Yes | | |
| Pump Force (Avg. Peak Load (N)) | | | | |
| Pump Output (Avg. Dose/Pump | | | | |
| (g)) | | | | |
| Foam Density (g/ml) | | | | |
| Appearance | Transparent | Transparent | | |

TABLE 5
Examples 18-23 of Shampoo Composition Examples

| | 18 | 19 | 20 | 21 | 22 | 23 |
|---|---|---|---|---|---|---|
| Disodium Laureth | — | — | — | — | — | — |
| Sulfosuccinate¹ | | | | | | |
| Lauramidopropyl Betaine² | 7.70 | 7.70 | 7.70 | 7.70 | 7.70 | 7.70 |
| Disodium Cocoyl Glutamate³ | 8.36 | 8.36 | 8.36 | 8.36 | 12.81 | 15.76 |
| Sodium C10-15 Pareth | — | — | — | — | 2.99 | — |
| Sulfate⁴ | | | | | | |
| Sodium Cocoyl Isethionate⁵ | — | — | — | — | — | — |
| Sodium Lauroyl Sarcosinate⁶ | — | — | — | — | — | — |
| Decyl Glucoside⁷ | 7.44 | 7.44 | 7.44 | — | — | — |
| Sodium Lauryl Sulfate⁸ | — | — | — | — | — | — |
| Linoleamidopropyl PG- | — | — | — | 7.44 | — | — |
| Dimonium Chloride | | | | | | |
| Phosphate⁹ | | | | | | |
| Stearamidopropyl | — | — | — | — | — | — |
| Dimethylamine¹⁰ | | | | | | |
| Propylene Glycol¹¹ | — | — | — | 5.32 | — | — |
| Dipropylene Glycol¹² | — | — | — | — | — | — |
| Sodium Xylene Sulfonate¹³ | — | — | — | — | — | — |
| Glycerin¹⁴ | 2.84 | 2.84 | 2.84 | 2.84 | 2.84 | 2.84 |
| Polyquat-76¹⁵ | 0.20 | 0.20 | 0.20 | 0.20 | 0.20 | 0.20 |
| Sodium Benzoate¹⁶ | 0.25 | 0.25 | 0.25 | 0.25 | 0.25 | 0.25 |
| Sodium Salicylate¹⁷ | 0.25 | 0.25 | 0.25 | 0.25 | 0.25 | 0.25 |
| DiSodium EDTA¹⁸ | 0.14 | 0.14 | 0.14 | 0.14 | 0.14 | 0.14 |
| Methylchloroisothiazolinone/ | — | — | — | — | — | — |
| Methylisothiazolinone¹⁹ | | | | | | |
| Piroctone Olamine²⁰ | 0.50 | 0.20 | 0.00 | 0.80 | 0.80 | 0.8 |
| Fragrance | 1.70 | 1.70 | 1.70 | 1.70 | 1.70 | 1.70 |
| Menthol²³ | — | — | — | — | — | — |
| Citric Acid²¹ | 0.76 | 0.76 | 0.76 | 0.76 | 0.76 | 0.86** |
| Salicylic Acid²² | 1.80 | 1.80 | 1.80 | — | — | — |
| Water | QS | QS | QS | QS | QS | QS |
| pH | 6.5-7.0 | 6.5-7.0 | 6.5-7.0 | 6.5-7.0 | 6.5-7.0 | 6.8 |
| Viscosity (cP) | | | | | | 9.30\hat{ }\hat{ } |
| Pumpable? | | | | | | Yes |
| Pump Force (Avg. Peak | | | | | | |
| Load (N)) | | | | | | |
| Pump Output (Avg. | | | | | | |
| Dose/Pump (g)) | | | | | | |
| Foam Density (g/ml) | | | | | | |
| Appearance | | | | | | Transparent, |
| | | | | | | light yellow |

*Viscosity (cP) @ 4 l/s; cup & bob
\hat{ }Viscosity (cP) @ 2 l/s; cone and plate
\hat{ }\hat{ }Viscosity (cP) @ 2 l/s; cup & bob
** Add as a 50% aqueous solution
   1. Disodium Laureth Sulfosuccinate, MACKANATE® EL P MB; Solvay
   2. Lauramidopropyl betaine, Mirataine® DAB ULS MB; Solvay
   3. Disodium Cocoyl Glutamate, AMISOFT® ECS-22W; Ajinomoto
   4. Sodium Deceth-1 Sulfate at 70% active, Procter & Gamble
   5. Sodium Cocoyl Isethionate, Hostapon® SCI-85 C; Clariant
   6. Sodium Lauroyl Sarcosinate, Hamposyl L-30-U; Chattem Chemical Inc
   7. Decyl Glucoside, Plantaren® 2000 N UP; BASF
   8. Sodium Lauryl Sulfate; Procter & Gamble
   9. Linoleamidopropyl PG-Dimonium Chloride Phosphate, Arlasilk™ EFA-LQ-(AP); Croda
   10. Stearamidopropyl Dimethylamine, Incromine™ SD-PA-(MH); Croda
   11. Propylene Glycol, Propylene Glycol USP/EP; Univar Solutions
   12. Dipropylene Glycol, Dipropylene Glycol Care; BASF
   13. Sodium Xylene Sulfonate, STEPANATE® SXS; Stepan
   14. Glycerin, GL99.7 USP; Cremer
   15. Polyquat-76, POLYQUATERNIUM-76; Solvay
   16. Sodium Benzoate from Emerald Kalama Chemical
   17. Sodium salicylate from JQC (Huayin) Pharmaceutical
   18. Disodium EDTA, Dihydrate (EDTA-2Na); Supreme Resources
   19. Kathon™CG, Methyl chloro isothiazolinone and Methyl isothiazolinone; DuPont
   20. Piroctone Olamine from Clariant Gmbh
   21. Citric Acid USP Anhydrous Fine Granular; Archer Daniels Midland
   22. Salicylic Acid USP; Novacyl
   23. Menthol Premix, 70% active; Procter & Gamble

For Example 3 in Table 1, it is determined that the slow addition of a 50% citric acid solution, in place of citric acid powder, resolves the issue of the formation of the white particles noted in the appearance description for Example 3 above.

From Table 2, Examples 6-9 are not pumpable. Example 6 is not attempted due to the formula having phase split. Example 7 is not pumpable due to high viscosity of the formulation. Examples 8 and 9 are not attempted due to significantly higher viscosities. From Table 3, Examples 10-12 are not pumpable. Example 10 is not attempted due to significantly higher viscosity than target. Examples 11 and 12 are attempted but are not pumpable due to high viscosities.

For Table 1, Table 2, Table 3. Table 4 and Table 5 the phase stability is determined as follows. The examples are prepared as described herein. The examples are put in a clear container with a lid attached finger tight. The examples are stored at ambient temperatures (20-25° C.), away from direct sunlight, and visually inspected the day of making and after 24 or 72 hours. The product is phase stable if by visual detection there is no phase separation, which includes precipitates, and the example appears homogeneous. As used herein, “visual detection” means that a human viewer can visually discern the quality of the example with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

For Table 1, Table 2, Table 3. Table 4 and Table 5 the pumpability are determined as follows. The examples are prepared as described herein. The examples are put into a new product package and the pump dispenser attached. Several attempts are made to prime the pump and dispense the product. If no or a poor quality foam is dispensed, or there is difficulty in dispensing or actuating the pump, a result of not pumpable is recorded. Foams dispensed that are of a slightly lower quality are also noted.

### ADDITIONAL EXAMPLES/COMBINATIONS

A. A compact, stable shampoo composition comprising:
   a. from about 18% to about 40%, by weight of the composition, of a surfactant system wherein the surfactant system comprises:
      i. from about 5% to about 40%, by weight of the composition, acyl glutamate;
      ii. from about 5% to about 40%, by weight of the composition, co-surfactant wherein the co-surfactant is selected from the group consisting of anionic surfactant, zwitterionic surfactant, and combinations thereof;
         wherein the shampoo composition is substantially free of sulfate-based surfactants;
         wherein the weight ratio of acyl glutamate to total surfactant is from about 0.30 to about 1;
   b. wherein the pH is from about 6 to about 8;
   c. wherein the composition has a viscosity of less than or equal to about 35 centipoise (cP).
B. A compact, stable shampoo composition according to Paragraph A, wherein the composition comprises from about 20% to about 30%, by weight of the composition, surfactant system.
C. A compact, stable shampoo composition according to Paragraph A-B, wherein the composition comprises from about 20% to about 30%, by weight of the composition, surfactant system.
D. A compact, stable shampoo composition according to Paragraph A-C, wherein the composition comprises from about 5% to about 16%, by weight of the composition, acyl glutamate.
E. A compact, stable shampoo composition according to Paragraph A-D, wherein a weight ratio of anionic surfactant to zwitterionic surfactant is from about 1.1 to about 15:1.
F. A compact, stable shampoo composition according to Paragraph A-E, wherein a weight ratio of anionic surfactant to zwitterionic surfactant is from about 1:1 to about 3:1.
G. A compact, stable shampoo composition according to Paragraph A-F, wherein the composition comprises from about 0.1% to about 0.8%, by weight of the composition, cationic polymer.
H. A compact, stable shampoo composition according to Paragraph A-G, wherein the shampoo composition comprises an organic solvent selected from the group consisting of propylene glycol, dipropylene glycol, alcohols, glycerin, and combinations thereof.
I. A compact, stable shampoo composition according to Paragraph A-H, wherein the shampoo composition is substantially free of a thickeners selected from the group consisting of acrylate polymers and co-polymers, xanthan gum, and combinations thereof.
J. A compact, stable shampoo composition according to Paragraph A-I, wherein the shampoo composition further comprises a conditioning agent selected from the group consisting of silicone conditioning agents, organic conditioning materials, and combinations thereof.
K. A compact, stable shampoo composition according to Paragraph A-J, wherein the shampoo composition further comprises scalp care active selected from the group consisting of piroctone olamine, azoles, salicylic acid, and mixtures thereof.
L. A compact, stable shampoo composition according to Paragraph A-K, wherein the acyl glutamate comprises disodium cocoyl glutamate.
M. A compact, stable shampoo composition according to Paragraph A-L, wherein the composition comprises a zwitterionic co-surfactant selected from the group consisting of cocamidopropyl betaine (CAPB), lauramidopropyl betaine (LAPB), and combinations thereof.
N. A compact, stable shampoo composition according to Paragraph A-M, wherein the shampoo composition has a viscosity from about 5 cP to about 30 cP.
O. A compact, stable shampoo composition according to Paragraph A-N, wherein the ratio of acyl glutamate to total surfactant is from about 0.2 to about 1.
P. A compact, stable shampoo composition according to Paragraph A-O, wherein the ratio acyl glutamate to total surfactant is from about 0.3 to about 1.
Q. A compact, stable shampoo composition according to Paragraph A-P, wherein a method of treating hair, the method comprising a) apply to the hair the stable shampoo composition according to Paragraph A-P, wherein the shampoo is dispensed from a mechanical foam dispenser as dosage of foam, and b) rinsing the hair composition.
R. A compact, stable shampoo composition according to Paragraph A-Q, wherein the foam has a density of from about 0.02 g/cm³ to about 0.3 g/cm³.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compact, stable shampoo composition comprising:
a. from about 18% to about 40%, by weight of the composition, of a surfactant system wherein the surfactant system comprises:
i. from about 5% to about 40%, by weight of the composition, acyl glutamate;
ii. from about 5% to about 40%, by weight of the composition, co-surfactant wherein the co-surfactant is selected from the group consisting of anionic surfactant, zwitterionic surfactant, and combinations thereof;
wherein the shampoo composition is substantially free of sulfate-based surfactants;
wherein the weight ratio of acyl glutamate to total surfactant is from about 0.30 to about 1;
b, wherein the pH is from about 6 to about 8;
c, wherein the composition has a viscosity of less than or equal to about 35 centipoise (cP).

2. The compact, stable shampoo composition of claim 1, wherein the composition comprises from about 20% to about 30%, by weight of the composition, surfactant system.

3. The compact, stable shampoo composition of claim 1, wherein the composition comprises from about 5% to about 16%, by weight of the composition, acyl glutamate.

4. The compact, stable shampoo of claim 1, wherein a weight ratio of anionic surfactant to zwitterionic surfactant is from about 1.1 to about 15:1.

5. The compact, stable shampoo of claim 1, wherein a weight ratio of anionic surfactant to zwitterionic surfactant is from about 1:1 to about 3:1.

6. The compact, stable shampoo composition of claim 1, wherein the composition comprises from about 0.1% to about 0.8%, by weight of the composition, cationic polymer.

7. The compact, stable shampoo composition of claim 1, wherein the shampoo composition comprises an organic solvent selected from the group consisting of propylene glycol, dipropylene glycol, alcohols, glycerin, and combinations thereof.

8. The compact, stable shampoo composition of claim 1, wherein the shampoo composition is substantially free of a thickeners selected from the group consisting of acrylate polymers and co-polymers, xanthan gum, and combinations thereof.

9. The compact shampoo composition of claim 1, wherein the shampoo composition further comprises a conditioning agent selected from the group consisting of silicone conditioning agents, organic conditioning materials, and combinations thereof.

10. The compact shampoo composition of claim 1, wherein the shampoo composition further comprises scalp care active selected from the group consisting of piroctone olamine, azoles, salicylic acid, and mixtures thereof.

11. The compact, stable shampoo composition of claim 1, wherein the acyl glutamate comprises disodium cocoyl glutamate.

12. The compact, stable shampoo composition of claim 1, wherein the composition comprises a zwitterionic co-surfactant selected from the group consisting of cocamidopropyl betaine (CAPB), lauramidopropyl betaine (LAPB), and combinations thereof.

13. The compact, stable shampoo composition of claim 1, wherein the shampoo composition has a viscosity from about 5 cP to about 30 cP.

14. The compact, stable shampoo composition of claim 1, wherein the ratio of acyl glutamate to total surfactant is from about 0.2 to about 1.

15. The compact, stable shampoo composition of claim 1, wherein the ratio acyl glutamate to total surfactant is from about 0.3 to about 1.

16. A method of treating hair, the method comprising:
a. applying to the hair the stable shampoo composition according to claim 1, wherein the shampoo composition is dispensed from an mechanical foam dispenser as a dosage of foam; and
b. rinsing the hair care composition.

17. The method of claim 16, wherein the foam has a density of from about 0.02 g/cm³ to about 0.3 g/cm³.
